(19) United States
(12) Patent Application Publication
(10) Pub. No.: US 2002/0012953 A1
(43) Pub. Date: Jan. 31, 2002

(54) Methods for separation and isolation of subpopulations of cells in biological samples

(76) Inventors: Eva Irene Stenbaek Jauho, Copenhagen (DK); Mogens Havsteen Jakobsen, Vanlose (DK)

Correspondence Address: EDWARDS & ANGELL, LLP, 130 Water Street, Boston, MA, 02109, US

(21) Appl. No.: 09/804,972

(22) Filed: Mar. 13, 2001

Related U.S. Application Data
(60) Provisional application No. 60/189,589, filed on Mar. 15, 2000.

(30) Foreign Application Priority Data
Mar. 14, 2000 (DK) PA 2000 00416

(51) Int. Cl. G01N 33/567; C12N 5/08

(52) U.S. Cl. 435/7.21; 435/366

(57) ABSTRACT

The present invention relates to novel methods for separation and isolation of sub-populations of cells in biological samples. In a presently preferred embodiment, the invention pertains to methods for separation, isolation and recovery of foetal cells from maternal blood by use of specific cell markers for specific subpopulations of foetal cells.

The present application claims the benefit of U.S. provisional application number 60/189589, which is incorporated herein by reference in its entirety.

### FIELD OF THE INVENTION

The present invention relates to novel methods for separation and isolation of sub-populations of cells in biological samples. In a presently preferred embodiment, the invention pertains to methods for separation, isolation and recovery of foetal cells from maternal blood by use of specific cell markers for specific subpopulations of foetal cells.

By the methods of the invention it is possible to isolate and detect even extremely small subpopulations of cells in a biological sample. These methods of the invention thus have a general application for isolation of small subpopulations of cells e.g. cancer cells, stem cells, malignant cells, mutated cells, blood cells, transfected cells, genetically modified cells, spermatocytes, cells from cell lines and cells at various differential stages in biological samples. These biological samples include blood, buffy coat, cerebrospinal fluid, urine, salvia and other biological fluid samples as well as tissue samples.

Important aspects of the invention relate to prenatal diagnosis and diagnosis of diseases as well as to identification of normal as well as malignant subpopulations of cells.

In a presently preferred embodiment, a non-porous body such as a functionally coated chamber slide with a Protein Immobilizer™ surface is used together with at least one protein e.g. an antibody, receptor or lectin with specificity for the cell population of interest. The cells bound to the non-porous body are preferably living cells and can be detached from the surface and grown in suitable media.

Furthermore, the present invention relates to a kit for use of the methods described above.

### BACKGROUND

Separation, isolation and recovery of cells are difficult tasks, especially when the concentration of relevant cells is extremely low compared to the total concentration of cells in the sample.

Many different methods for separation, isolation and recovery of cells are being used today, e.g. Fluorescence Activated Cell Sorter (FACS) (Lewis et al. 1996, Sohda et al. 1997, Jonker et al. 1997), magnetic beads separation (Ganshirt-Ahlert et al. 1992), gradient centrifugation (Oosterwijk et al. 1996), flow cytometri (Maino et al. 1998, Price et al. 1991, Tse et al. 1994), Wang tube technology (Wang et al. 1995), culturing of cells (Valerio et al. 1996), and panning (Okarma et al. 1992). These methods are often methods that require expensive equipment, e.g. FACS, are time consuming and give very low recovery of cells. Moreover, these methods all have the disadvantage of rupturing the cells to some extent and give a low recovery.

Separation and isolation of foetal cells is at present performed by invasive sampling e.g. chorionic villus sampling and amnionic villus sampling (Bianchi et al. 1995). These methods include a high risk of abortion (1%) and can only be performed at a certain period during pregnancy.

The abortion limit in Denmark is 12 weeks of pregnancy. For chorionic villus sampling the samples are taken after week 10 and for amnionic villus sampling the samples are taken after week 15. These samples are therefore taken at a very late stage of the pregnancy. By blood sampling the blood samples may be taken non-invasively as early as 6 weeks of pregnancy (Bianchi et al. 1998).

There is a general need for a gentle and simple method of isolation and separation of subpopulations of cells in biological samples, e.g. foetal cells of interest in mothers' blood.

### SUMMARY OF THE INVENTION

The invention provides new methods for separation, isolation and recovery of sub-populations of cells in biological samples.

The invention further provides methods for separation, isolation and recovery of a subpopulation of cells in a biological sample by using a ligand suitable for detection of the desired cell type bound regiospecifically to the surface of a non-porous body such as a slide with a Protein Immobilizer™ surface.

The invention further provides methods for separation, isolation and recovery of foetal cells from maternal blood by use of specific cell markers on specific subpopulations of foetal cells.

The invention also provides methods of separation, isolating and recovery of foetal cells in a biological sample using a monoclonal or polyclonal antibody reacting specifically with a cell marker which is specific for human foetal cells or using a receptor specific for a ligand on the human foetal cell surface.

The present invention has a general application for isolation of small subpopulations of cells e.g. cancer cells, stem cells, malignant cells, mutated cells, blood cells, spermatocytes, cells from cell lines and cells at various differential stages in biological samples. The different cell types may be isolated from biological samples including blood, buffy coat, cerebrospinal fluid, urine, salvia, tissue samples, other biological fluid samples and tissue samples.

The invention also provides kits for use in the methods described herein.

Methods of isolation and separation of cells by use of specific cell markers on the required cell surface include techniques such as binding of cells to magnetic beads, flow cytometri and FACS. In general many different cell markers may be used for isolation of cells. These include: CD-antigens, cell surface receptors (e.g EPOr, T cell receptor), genetic markers (HLA antigens), specific outer membrane proteins (e.g. OMPs, hemolysins, toxins, N-CAM, I-CAM, cadherins and transporting proteins), specific cell surface carbohydrates and surface immunoglobilins (sIg). These markers can be found on many different cells types and subtypes e.g. cancer cells, stem cells, malignant cells, mutated cells, blood cells, spermatocytes, cells from cell lines and cells at various differential stages in biological samples (Gage et al. 1995, Maino et al. 1998, Aglietta et al. 1998, Holm et al. 2000, Olesen et al. 2000, Bidart et al. 1999, Davis et al. 1999, Reilly et al. 1996, Looker et al.1999, Bertolini et al. 1997, Cha et al. 1997).

Foetal cells in maternal blood consist of many subpopulations of cells and have many different cell markers on the cell surface, among these are CD71. Of the foetal cells, erythroblasts are the most relevant cell to detect in maternal blood samples as they have CD71 on the cell surface, they contain foetal haemoglobins and have a nucleus. CD71 is the transferrin receptor, which binds transferrin in the blood. Transferrin is an important iron binding and transporting protein in blood. CD71 is also found in circulation in the blood. Erythroblasts can be detected very early in maternal blood and are relative numerous among the foetal cell population in maternal blood throughout the pregnancy.

Other cell types such as trophoblasts or lymphocyte subpopulations have also specific cell markers suitable for foetal cell isolation (Bertero et al. 1988, Covone et al. 1984).

It has been shown that cells early in erythroid cell line development have a higher concentration of CD71 on the cell surface that cells later in the erythroid cell line development. Hence, erythroblasts, which are early in the erythroid cell line development, may therefore have a higher concentration of CD71 on the cell surface (Looker et al. 1999). It has also been shown that CD71 in circulation is higher in foetuses than in adults (Skikne et al. 1988) and that the concentration of circulating CD71 correlates to the concentration of cell surface CD71. Erytroblasts from foetus blood may be found in maternal blood as early as 6 weeks of pregnancy (Bianchi et al. 1998). Previously, several groups have shown that foetus cells enter the blood stream of the mother as early as in the 4th week of the pregnancy. Furthermore, it has been shown that cells early in the erythroid cell line differentiation have a higher density of CD71 on the cell surface (Beguin et al. 1993). Thus, CD71 is a well-suited cell marker for the detection of foetal cells in the maternal blood samples.

A number of commercially available non-porous bodies, e.g. surface-modified slides, can be used to immobilize proteins, including antibodies, receptors, and/or lectins. However, use of the generally available surfaces mentioned above often creates background problems, especially when complex mixtures are analysed. A significant decrease in the background has been obtained when the ligand is covalently attached to a solid surface by the anthraquinone (AQ) based photo-coupling method described in e.g. WO 96/31557. This method allows the covalent attachment of the desired relevant molecules in a biological sample to the surface of a non-porous body.

The principle in the AQ-technology, which is a photochemical method of immobilising ligands on carbon containing material surfaces, where the ligands are not subjected to damaging treatments and therefore substantially maintain their functions, even when the ligands are sensitive biomolecules, is described in detail in WO 96/31557.

On the basis of the AQ-technology, the Protein Immobilizer™ technology has been developed for covalent binding of proteins, such as enzymes, lectins, receptors and antibodies, to microtiter plates, petri dishes, tubes, membranes, frits and slides. Products prepared on the basis of this technology are commercially available from Exiqon, Vedbaek, Denmark. A Protein Immobilizer™ surface is produced by photochemical coupling of a thermochemically active molecule to the surface. The characteristics of the Protein Immobilizer™ surface are high binding capacity of proteins, high reproducibility and uniformity of the bound proteins. Furthermore, the Protein Immobilizer™ surface is stable at room temperature over long periods of time.

Using this technology it has been possible to develop a Protein Immobilizer™ coated chamber slide with Protein Immobilizer™ surfaces and properties. Protein Immobilizer™ surface is able to bind proteins e.g. antibodies, enzymes, receptors and lectins. The relevant antibodies, directed against e.g. foetal cell markers, are bound regio-specifically to the chamber slides by means of this functional surface. These functional chamber slides are able to specifically bind even very small subpopulations of foetal cells from maternal whole blood samples. Cells isolated on chamber slides can be used directly for prenatal genetic diagnostics, e.g. when using fluorescence in situ hybridisation (FISH).

Furthermore, the isolated cells may be cultivated directly in the chamber slide or the cells can be removed and cultivated. This type of diagnostic assay will make the access of samples (blood sample) risk free as regards to abortion, shorten the time of analysis and avoid expensive equipment, thereby making it less expensive. Moreover, the test may be performed sooner during the pregnancy and for a longer time period of the pregnancy.

Using the correct cell markers, other subpopulations, e.g. cancer cells, stem cells, malignant cells, mutated cells, blood cells, spermatocytes, cells from cell lines, cells at various differential stages and other cell populations, may be isolated in a similar manner, e.g. from biological fluid samples or biopsies. Isolation of a specific cell population may be used for positive isolation (isolation of wanted cells) or for negative isolation of cells (isolation of unwanted cells).

The obtained isolated cells may be used for genetic or immunological diagnosis/analysis, or for further cell growth for use in e.g. transplantation. Moreover, genetic analysis of isolated cells may be performed e.g. analysis for successful transformation of cells from transgenic mice, analysis for successful incorporation of genes by using antisense probes.

For immunological analysis, cell surface and cytoplasmic markers can be detected by Enzyme Immunoassay (EIA).

By the methods of the invention, many more samples can be run daily than by the present methods, as many samples can be run in parallel and not be run in series as in other methods e.g. FACS. Further, the methods of the invention can be performed as a one step procedure and do not require expensive equipment. Finally, the methods provide separation, isolation and recovery of the desired cells with a very high recovery rate and noticeably, a substantial number of the cells are viable.

In the following glossary list, the frequently used abbreviations are shown.

GLOSSARY

| | |
|---|---|
| AQ | Anthraquinone |
| AVS | Amnionic Villus Sampling |
| BSA | Bovine Serum Albumin |
| BSA/SSC | Bovine Serum Albumin/Saline Sodium Citrate |
| CBS | Cord Blood Sample |
| CD | Cluster differentiation |
| CVS | Chorionic Villus Sampling |
| DAPI | 4′,6-diamidino-2-phenylindole |
| EIA | Enzyme Immunoassay |
| EPOr | Erythropoietin receptor |
| FACS | Fluorescence Activated Cell Sorter |
| FISH | Fluorescence In Situ Hybridisation |
| FITC | Fluorescense isotiocyanate |
| NP | Nonylphenoxy Polyethoxy ethanol |
| OPD | Ortho Phenylin Diamine |
| PBS | Phosphate Buffer Saline pH 7.2 |
| PBS-T | Phosphate Buffer Saline-0.05% Tween 20 |
| PI | Protein Immobilizer ™ |
| SSC | Saline sodium citrate |
| SSC-T | Saline Sodium Citrate-0.05% Tween 20 |
| TRITC | Tetramethylrhodamine B isothiocyanate |
| W | Pregnant Women |

### DESCRIPTION OF THE DRAWINGS

FIG. 1: Schematic representation of a chamber slide with one chamber (24×48 mm): X) chamber; Y) slide. The area for cell binding on the chamber slide is grey. The chamber is open at the top for sample application.

FIG. 2: Schematic representation of protein binding to the Protein Immobilizer™ surface: A) Protein Immobilizer molecule; E) Receptor; F) Antibody; G) Lectin.

FIG. 3: Binding capacity of glass and of permanox chamber slides with and without Protein Immobilizer™ (PI) surface. White bar: With PI; Black bar: Without PI. Y-axis: OD492 nm.

FIG. 4: Schematic representation of the lack of binding a foetal cell (D) to a CD71 antibody (C), which is directly bound to the Protein Immobilizer molecule (A) on the chamber slide.

FIG. 5: Schematic representation of a foetal cell (D) bound to a CD71 antibody (C), which is bound to an antibody against Ig (Fc-part) (B), which again is bound to the Protein Immobilizer (A) molecule on the chamber slide.

FIG. 6: Effect of coupling of goat anti-mouse IgG (Fc-part) in 0.1M sodium phosphate pH 8.0 or in 0.1M sodium carbonate pH 9.6 on glass and permanox chamber slides. Black bar: pH 8.0; White bar: pH 9.6. Y-axis: OD492 nm.

FIG. 7: The biological variation of bound DAPI (4′,6-diamidino-2-phenylindole) positive cells from 26 cord blood samples. X-axis: Cord blood sample number; Y-axis: Number of cells per mm².

FIG. 8: The variation of bound foetal cells in foetus blood samples (DAPI positive cells per mm²). The blood samples were drawn from foetuses of different age (weeks of pregnancy). X-axis: Age of foetus (weeks); Y-axis: Number of cells per mm².

FIG. 9: Binding of maternal cells from 27 maternal blood samples. X-axis: 2 Maternal blood sample number; Y-axis: Number of cells per mm .

FIG. 10: Two-fold titration of a cord blood sample (boy) in the respective maternal blood sample (titration range: 1:0 to 1:7). X-axis: The relative concentration of the two blood samples (cord blood: maternal blood); Y-axis: Number of cells per slide. White bar: XX-cells; Black bar: XY-cells.

FIG. 11: Four-fold titration of a cord blood sample (boy) in the respective maternal blood sample (titration range 1:0 to 1:16.383). The relative concentration of the two blood samples (cord blood: maternal blood); Y-axis: Number of cells per slide. White bar: XX-cells; Black bar: XY.

FIG. 12: Schematic representation of a foetal cell (D) bound to a CD71 antibody (C) and subsequently detached from the chamber slide. B) Antibody against Ig (Fc-part); A) Protein Immobilizer molecule; H) Detachment of the cell by e.g. laser, enzymatic reaction, chemical reaction or by chemical modification of anti-CD71 molecule.

### DETAILED DESCRIPTION OF THE INVENTION

The present invention relates to a method for separation and isolation of a desired cell type in a biological sample from e.g. a mammal such as a human being. By the method of the invention it is possible to isolate and detect even extremely small subpopulations of cells in a biological sample.

In particular, the present invention provides methods for separation and isolation of cells having a high recovery rate and a high purity. The methods of the invention are useful for separation and isolation of living cells as the methods are without any or minimal rupture of the cells. After the separation and isolation the cells are generally in a condition suitable for further diagnosis and analysis e.g. by optical detection, genetic and/or protein analysis. The purity of the obtained isolated cell populations is generally adequate for further cell culture.

The following detailed description of presently preferred embodiments of the invention will focus on separation, isolation and recovery of foetal cells from maternal blood by use of a specific cell marker such as CD71 for a specific subpopulation of foetal cells. It will be appreciated, however, that all disclosures relating to separation, isolation and recovery apply also to other cell markers, as the person of skill in the art on the basis of the disclosure of the inventive concept will be able to make modifications as appropriate.

Hence, the separation, isolation and recovery of foetal cells by use of CD71 only exemplifies the invention, but other subpopulations of cells e.g. cancer cells, stem cells, malignant cells, mutated cells, blood cells, transfected cells, genetically modified cells, spermatocytes, cells from cell lines and cells at various differential stages in biological samples can be separated and isolated by use of other specific cell markers in the embodiments described hereinafter.

The generic design of the Protein Immobilizer™ microtiter plates has served as the basic set up for the development of the non-porous body suitable for optical detection and analysis. Examples of such bodies are a sheet, a film, a disc, a plate, a ring, a rod, a tube, a tray, a tube, a microtiter plate, a cluster tray, a stick and a slide, such as a microscope slide. In presently preferred embodiments, the body is substantially flat at least in the area wherein the optical detection or analysis is to take place.

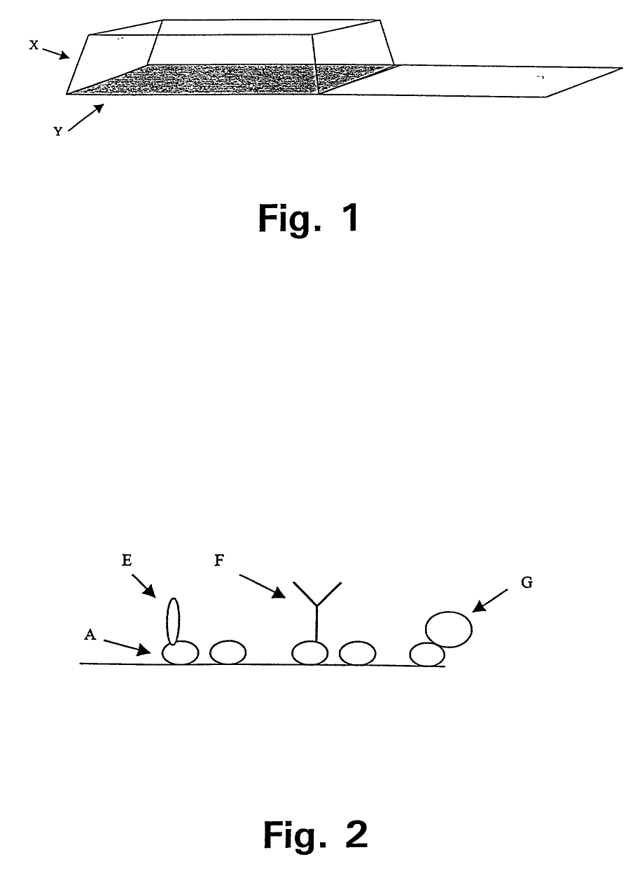

In a preferred embodiment, the substantial non-porous body is a functional chamber slide. These functional chamber slides can bind proteins, e.g. antibodies, receptors and lectins. The set up of the functional surface is such that the cell marker specific antibodies are bound regio-specifically to the chamber slide. An antibody against Ig (Fc-part) is thus first bound to the chamber slide followed by binding of an antibody against a cell marker. A relevant cell with the specific cell marker on cell surface will thus have optimal conditions for binding to the antigen binding site of the antibody against cell marker. The chamber slides used are chamber slides with one or two chambers. A one-chamber slide is outlined in FIG. 1.

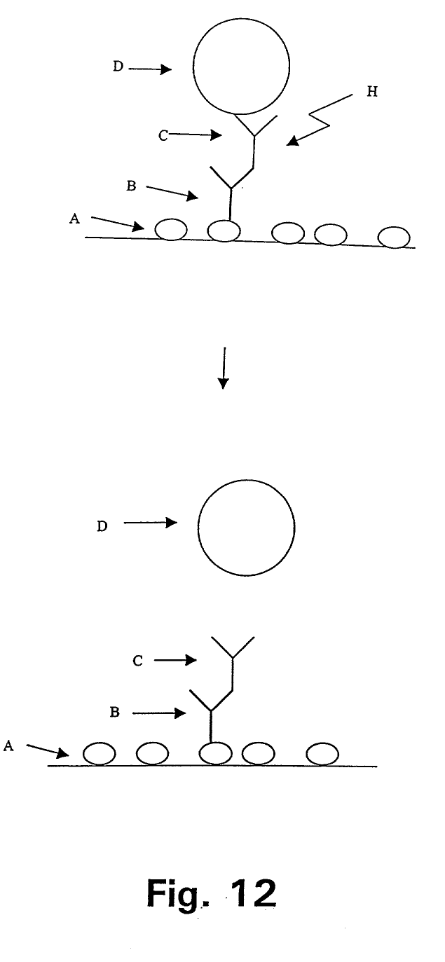

In a further embodiment of the present invention a relevant cell is bound to the chamber slide as described above and subsequently detached from the chamber slide. Detachment of the relevant cell from the chamber slide may be performed by means of e.g. a laser, an enzymatic reaction, a chemical reaction or by chemical modification of the antibody against a cell marker (see FIG. 12).

Below the immunochemical design and optimisation of the functional chamber slides is described. For the development of a suitable functional surface for the separation of cells from whole blood, commercially available chamber slides can be used as starting material. Using the Protein Immobilizer™ technology described in WO 96/31557, functional chamber slides have been developed. These functional chamber slide surface are able to bind proteins, including antibodies, receptors, and/or lectins to the surface. The generic method for binding of proteins to chamber slides is outlined in FIG. 2.

The immunochemical development and optimisation work described in further detail below was done using 8-chamber slides (glass (177380, Nunc) and permanox (177429, Nunc)).

It is possible to make a similar development using other sources of bodies for isolation, separation and recovery of desired cells. The following comments shall therefore be used as a guidance for the person skilled in art aiming at developing other functional surfaces or use the described functional surfaces for isolation of other desired cell populations.

To the functional chamber slides specific antibodies directed against specific foetal cell markers were bound in such a manner that the antibody surface could be used for separation of subpopulations of cells.

Generally, all immunochemical optimisation took place according to the following enzyme immunoassay (EIA) protocol on chamber slides with a functional Protein Immobilizer™ surface:

Enzyme immunoassay (EIA) protocol (an outline):

1. Incubation with antibody directed against Ig (the Fc-part). Wash 3× in PBS-T (phosphate buffered saline with Tween 20) after incubation.

2. Incubation with antibody directed against cell markers. Wash 3× in PBS-T after incubation.

3. Incubation with horse radish peroxidase (HRP)-conjugated antibody directed against the cell marker antibody. Wash 3× in PBS-T after incubation.

4. Substrate-chromogene solution (Ortho Phenylene Diamine (OPD)) was added. The reaction was stopped after 10 minutes with sulphuric acid. The final solution (200 μl per chamber slide) was transferred to a microtiter plate and analysed on an enzyme-linked immunosorbent assay (ELISA) reader.

General remarks concerning the development of the following procedures A) Protein Immobilizer™ surface, B) immunochemical parameters, C) incubation of cells and D) cell assays are outlined below:

A) The Protein Immobilizer™ Surface

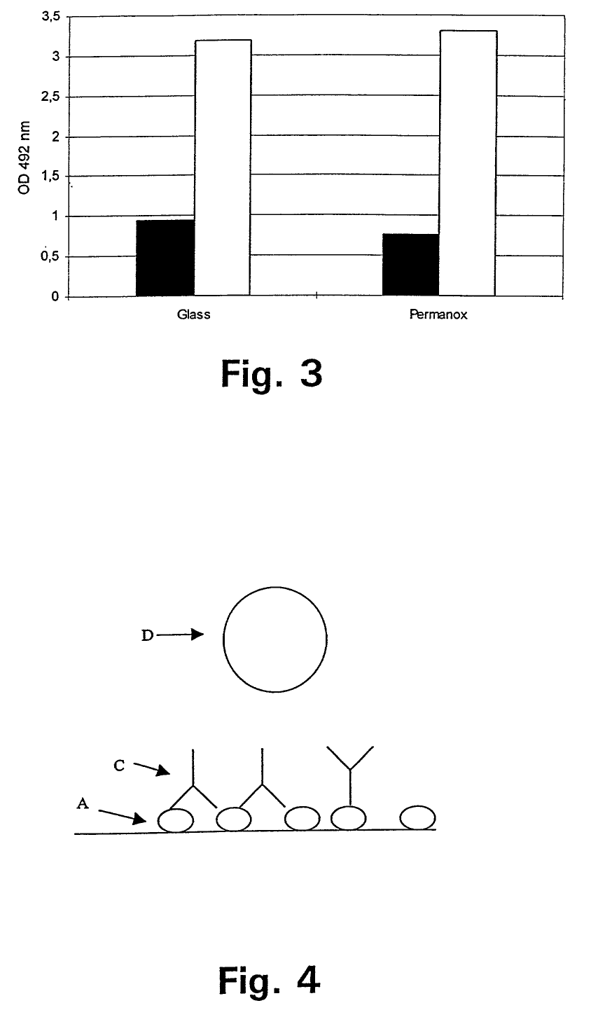

8-chamber slides were used (glass and permanox) for optimisation. The functional Protein Immobilizer™ surface was optimised as regards to the concentration of Protein Immobilizer™ and UV illumination time (see example 1). Subsequently, the assay conditions were transferred to 1-chamber and 2-chamber slides and tested both immunochemically and in the cell assay (FIG. 3) (see example 2).

The optimised parameters are shown in Table 1.

B) Optimisation and Testing of the Immunochemical Parameters

i) Method for Producing the Antibody Surface on the Chamber Slide Two methods for the application of antibody were tested (see example 1):

Method 1: Antibody against cell markers was applied to the chamber slides.

Method 2: Antibody against Ig (the Fc-part of an immunoglobulin) was first applied to the chamber slides, secondly an antibody against a cell marker was applied. Thus, the antibody against a cell marker was bound in such a manner that the antigen binding sites were situated outwards from the chamber slide surface.

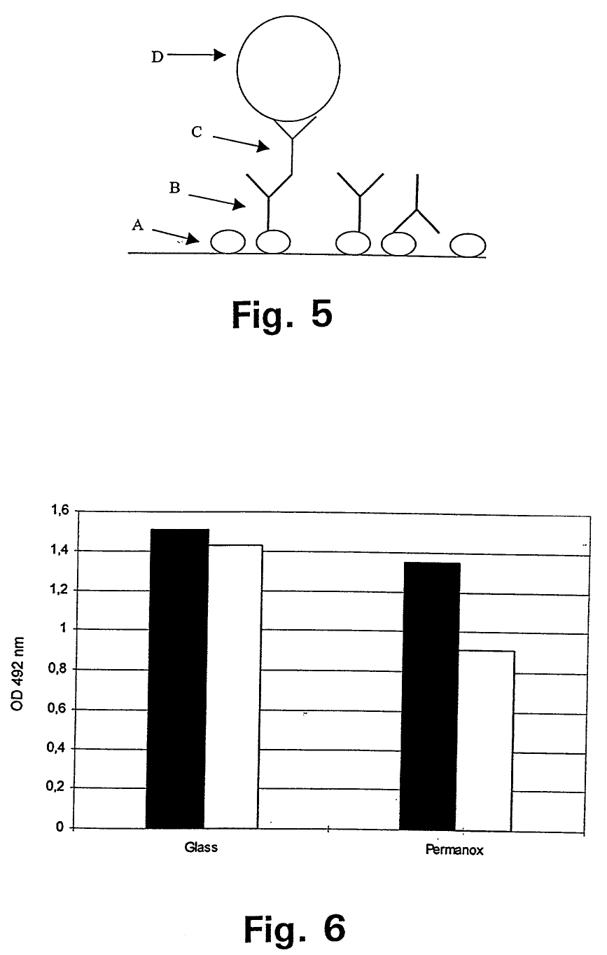

Both methods were tested in a cell assay (example 2). For separation of foetal cells method 1 bound no or very few cells, and method 2 bound a lot of cells (see examples 2 and 6). Thus, it was important that the antibody directed against a cell marker was bound regiospecifically to the chamber slide surface so that the antigen binding site of the bound antibody was directed against the cell surface. In the following only Method 2 was used for preparation of antibody bound chamber slides. The binding of cells to the chamber slides by the two methods are illustrated in FIG. 4 and 5.

ii) Optimisation of Assay Parameters

For the optimisation of the antibody against Ig (Fc-part), the following parameters were optimised (see example 1):

Species specificity of antibody.

Antibody concentration.

Coupling buffer for antibody.

Incubation time of antibody.

When optimising the antibody against Ig (Fc-part) (first antibody) and the antibody against cell markers (second antibody), the species of the antibodies were chosen in such a manner that the antibodies would not interfere with the antibodies in the following cell assay. The binding capacity of the antibody against Ig (Fc-part) to the Protein Immobilizerm surface was studied after dilution in various buffers. The antibodies were tested in various concentrations. Furthermore, the incubation times were also optimised.

It was investigated whether it was necessary to block the anti-Ig (Fc-part) coated chamber slides.

When optimising antibody against cell markers, each antibody was optimised individually. For the antibody against CD71, the antibody concentration and the incubation time were optimised. This was likewise optimised for the antibody against EPOr and for the antibody against CD36. Furthermore, various antibodies against EPOr were tested, including both polyclonal and monoclonal antibodies.

The optimum functional antibody surface was established from optimising in EIA as described above on 8-chamber slides. Subsequently, the assay conditions were transferred to 1-chamber and 2-chamber slides and tested both immunochemically and in the cell assays (see example 2).

The optimised parameters are shown in Table 1.

C) Incubation of the Cells (the Cell Application)

The functional anti-CD71 chamber slides were incubated with blood samples. All blood samples used in the cell assay were used within 20 hours after they had been drawn and were kept in a refrigerator until use.

The dilution of blood samples, dilution buffer, incubation time, incubation temperature and application method were optimised. For optimerisation of all these parameters, cord blood samples were used (see examples 3 and 5). After incubation of the blood samples the chamber slides were washed in an appropriate buffer. The bound cells on the chamber slides were fixed by various standard fixation procedures, dehydrated and dried (see example 3). The chamber slides were now ready for the cell assay.

D) The Cell Assay

The cell assays on chamber slides were carried out according to the manufacturers standard protocol. 1-chamber and 2-chamber slides were used. The detection of specific subpopulations of cells bound to the chamber slides was performed using one or a combination of the following three assays:

1. Staining with 4′,6-diamidino-2-phenylindole (DAPI) (binds DNA in the nucleus).

2. Detection of gamma-haemoglobin in the cytoplasm by use of specific gamma-haemoglobin antibodies.

3. In situ hybridisation of X- and Y-chromosomes using fluorescence in situ hybridisation (FISH).

The number of cells bound to the chamber slides was counted in a fluorescence microscope and was given in number of cells per slide or per mm² of the slide.

General remarks concerning the parameters analysed in a) Protein Immobilizer™ surface, B) immunochemicalparameters, C) incubation of cells and D) cell assays are outlined below:

All parameters in the assays were optimised in accordance with the above mentioned immunochemical assay (EIA) protocol.

A) The Protein Immobilizer™ Surface

8-chamber slides were used (glass and permanox) for optimisation. The functional Protein Immobilizer™ surface was optimised as regards to the concentration of Protein Immobilizer™ and UV illumination time. It was determined that the optimum UV illumination time was 5 minutes.

B) Optimisation and Testing of the Immunochemical Parameters

The antibody against Ig (Fc-part) (first antibody) and the antibody against cell markers (second antibody) were optimised.

For the anti-Ig (Fc-part) antibodies, different species (goat and rabbit), coupling buffers, incubation temperature and incubation time were investigated. The buffers 0.1M sodium carbonate pH 9.6, 0.1M sodium phosphate pH 8.0, 0.1M sodium bicarbonate pH 8.1 or PBS pH 7.2 were used.

This investigation showed that goat anti-mouse IgG (Fc), diluted 1:800 in 0.1M sodium phosphate buffer pH 8.0, was the optimum conditions for both glass and for permanox chamber slides (FIG. 6). The chamber slides were incubated overnight at 4° C. with agitation (100 rpm).

It was examined whether it was necessary to block the anti-Ig (Fc-part) coated chamber slides. Active blocking with 1% glycine or 1% lysine (the NH₂-group could be actively bound to the Protein Immobilizer™ surface) and passive blocking with 1% BSA were examined. It was found that blocking the functional surface on the chamber slides was not necessary.

When optimising antibody against cell markers, each antibody was optimised individually. The concentration of the antibodies and incubation time was optimised. The optimal conditions for antibody against CD71 was 1:1000 diluted in PBS-T and incubation for one hour at room temperature with agitation. These conditions applied to both glass and permanox slides.

In conclusion it appears when testing the various parameters that the optimum conditions for 8-chamber slides were optimum both for 1-chamber and for 2-chamber slides (both for glass and permanox). The following assay conditions are determined from the above-mentioned optimisation:

First antibody (Antibody directed against the Fc-part of an immunoglobulin)

Goat anti-mouse IgG (Fc), dilution 1:800 in 0.1M sodium phosphate pH 8.0.

Overnight incubation at 4° C. Wash 3× in PBS-T.

Blocking of the slides was unnecessary.

The chamber slides were dried with nitrogen if they were to be stored.

Second antibody (Antibody directed against the cell marker)

Mouse anti-human CD71 dilution 1:1000 in PBS-T.

Incubation for one hour at room temperature. Wash 3× in PBST.

Subsequently, the chamber slides were ready for the cell application and the cell assay.

C) Incubation of the Cells (the Cell Application)

The functional anti-CD71 chamber slides were incubated with blood samples. The blood samples (heparin blood) dilution, the dilution media, incubation time, incubation temperature and flow application method were investigated. One ml diluted blood samples were applied to 2-chamber slides and 2.5 ml diluted blood samples were applied to 1-chamber slides. Blood samples were incubated with agitation. After incubation the chamber slides were washed using various washing procedures.

The bound cells on the chamber slides were centrifuged (cytospin) and subsequently fixed using various fixation procedures, dehydrated and dried. The chamber slides were now ready for cell assay.

The following incubation conditions were found to be optimal:

1:4 dilution of blood samples

dilution of blood samples in PBS (RHONE)

1 hour incubation time

20-25° C. incubation temperature

flow application of blood samples

careful washing in PBS

cytospin not necessary

fixation in 2% paraformaldehyde in PBS for 5 minutes

The optimisation of blood sample incubation was carried out on cord blood samples. The types of blood samples tested using these optimal conditions were: cord blood, foetus blood, blood samples from pregnant women and blood samples from women after labour (see examples 6, 7 and 8).

D) The Cell Assay

The detection of specific subpopulations of cells bound to the chamber slides were performed using one or a combination of the following three assays. 1-chamber and 2-chamber slides were used.

1. DAPI staining: DAPI stain containing Vectashield was added to each slide. Two cover slips (18×18 mm) were applied to each slide (both for 1-chamber and 2-chamber slides).

2. Gamma haemoglobin detection: The slides were blocked and antibody against gamma haemoglobin applied. The slides were incubated for 30 minutes with agitation. The slides were washed. Donkey anti-sheep Ig conjugated with Tetramethylrhodamine B isothiocyanate (TRITC) was added to the slide. The slides were incubated for 30 minutes with agitation in the dark. The slides were washed with agitation in the dark. The slides were assembled with DAPI.

3. Fluorescence in situ hybridisation of X- and Y-chromosomes: Hybridisation mix containing XY-probes (DNA) was applied to each slide area (for 1-chamber slide 2×9 μl is applied). The X-probe was conjugated to TRITC and the Y-probe was conjugated to FITC. The slides were assembled with a cover slip and hybridazed in an incubator overnight. The slides were washed (in the dark) and subsequently dried in the dark. The slides were assembled with DAPI.

The cell count of cells bound to the chamber slides was performed using fluorescence microscopy. The cell count was either stated per mm² or per slide. All blood samples used were used within 20 hours after they had been drawn.

The number of cells bound to the chamber slides was counted in a fluorescence microscope and was given in number of cells per slide or per mm² of the slide. All blood samples used in the cell assay were used within 20 hours after they had been drawn and were kept in a refrigerator until use.

Thus, the present invention relates to separation and isolation of a desired cell type in a biological sample from a mammal or a human by binding said cell to a ligand specifically recognising said cell, the method comprising binding the ligand regiospecifically to the surface of a substantially flat non-porous body suitable for optical detection and analysis.

Hence, the invention relates to a method for separation, isolation and recovery of subpopulations of cells in a biological sample.

By the term “separation and isolation” is herein understood a separation of a sub-population of desired cells from a total population of cells and further, if desired, an isolation of the desired cells which are bound to the body.

By the term “desired cell type in a biological sample” is herein understood the cell type selected for separation and isolation in a biological sample. Examples of a desired cell pertain to a foetal cell, blood cell, cancer cell, stem cell, malignant cell, mutated cell, transfected cell, genetically modified cell, spermatocyte or a cell from a cell line. The stem cell may be a hematopoietic stem cell giving rise to e.g. the following progenitor cells: T cell progenitor, B cell progenitor, granulocyte-monocyte progenitor, eosinophil progenitor, basophil progenitor, megakaryocyte and erythroid progenitor, The desired cell may also be a cell mentioned above at a specific differential stage. Furthermore, the desired cell may be a blood cell at a specific differential stage of the myeloid or lymphoid cell lineage.

In an important aspect the invention relates to prenatal diagnosis, diagnosis of diseases, and identification of normal as well as malignant subpopulations of cells.

The ligand may be a polypeptide, a hormone or a polysaccharide or a protein. Examples of protein ligands are: an antibody, a receptor, an enzyme and a lectin. The antibody (monoclonal and/or polyclonal) may be specific for a cell surface marker, cell surface receptor or a MHC cell surface antigen on the respective cell.

Examples of cell markers pertain to CD-antigens (e.g. CD71), cell surface receptors (e.g. EPOr, T cell receptor), genetic markers (HLA antigens), specific outer membrane proteins (e.g. OMPs, hemolysins, toxins, N-CAM, I-CAM, cadherins and transporting proteins), specific cell surface carbohydrates and surface immunoglobilins (sIg).

By the term “regiospecific” is herein meant an orientation of the ligand whereby the binding site of the ligand exhibits the optimal orientation towards the cell and thus provides maximal binding between cell and ligand, e.g. between the specific cell marker and the cell in question. For binding of antibodies this means that the antigen binding site of the antibody is fully orientated towards the cell marker antigen. It is very important that the antibodies attached to the body are bound regiospecifically in order to achieve maximal binding capacity to the relevant cells. Using the described set up even very small subpopulations of cells can be specifically bound to the body.

By the tem “non-porous body” is meant that fluids and cells do not enter or pass through the surface of the body. By the term “substantially flat” is meant that in practical terms it should be possible to perform optical detection and analysis of cells bound to the “non-porous body”. It is understood that the maximum surface height variation across the (at any time) investigated area should be smaller than or equal to the “focal depth” of the applied optical detection method. By “focal depth” is understood the maximum surface height variation of an investigated surface accepted by the chosen optical detection method in order to achieve reliable results. Typically the maximum surface height variation should be 5 per thousand or less of the extend of the investigated area. However, this will depend on the body and the detection mechanism. Examples of optical detection methods include light microscopes, fluorescence microscopes, confocal microscopes and scanners, laser scanner devices, CCD based imaging and scanning devices, fiber optic based imaging and scanning devices.

By the term “suitable for optical detection and analysis” is herein understood that the substantially flat non-porous body can be used in optical imaging and scanning devices including, but not limited to, light microscopes, fluorescence microscopes, confocal microscopes and scanners, laser scanner devices, CCD based imaging and scanning devices, fiber optic based imaging and scanning devices.

The substantially flat non-porous body may be made from plastic, glass, silicone, silicone oxide (silica) or a composite material thereof. The body may also be a polystyrene, polyethylene, polyvinylacetate, polyvinylchloride, polyvinylpyrrolidone, polyacrylonitrile, polymethyl-methacrylatepentylene, polyester, polypropylene, polyvinylidendifluoride, polycarbonate or Topas™ surface.

Examples of a substantially flat non-porous body pertain to a sheet, a film, a disc, a plate, a ring, a rod, a tube, a tray, a microtiter plate, a cluster tray, a stick, a slide and a microscope slide.

The biological sample may be obtained from an individual such as a mammal including a mouse, a rat, a guinea pig, a pig, a rabbit, a monkey, a cat, a dog, a horse, a cow, a goat, a sheep and a human. However, tissue from other mammals may be useful according to the present invention.

Examples of biological samples pertain to blood, buffy coat, cerebrospinal fluid, urine, salvia and other biological fluid samples. The biological sample may also be a tissue sample.

The individual may be a healthy individual, an individual considered to have a risk of suffering from a disease or an individual suffering from a disease. Furthermore, the individual may also be an individual who has suffered from a disease and is therefore of interest to determine whether the desired cell type is present in the sample. The individual may also be a dead individual.

By the term “considered to have a risk of suffering from a disease” is herein understood that the individual has a higher/increased risk of suffering from the diseases compared to a healthy individual. The increased risk of suffering from the diseases may e.g. be due to a hereditary predisposition.

Examples of diseases include, but are not limited to, cancer, arthritis, allergy, inflammatoric disorders or neurological disorders.

Examples of biological samples pertain to blood, buffy coat, cerebrospinal fluid, urine, salvia and other biological fluid samples. The biological sample may also be a tissue sample.

Examples of assays for detection of specific sub-populations of cells bound to the body in the methods of the present invention include FISH, antibody-based detection and histochemical staining.

Examples of detectable labels which can be used in the methods of the present invention include radioisotopes, fluorochromes (e.g. TRITC, FITC, Texas red, Rhodamine, Cascade, Blue, Isosulfan blue, Oregon Green, Rhodol Green) or enzymes such as acetylcholinesterase, alkaline phosphatase, α-glycerophosphatase dehydrogenase, asparaginase, β-galactosidase, β-V-steroid isomerase, catalase, glucoamylase, glucose oxidase, glucose-6-phosphate dehydrogenase, horse radish peroxidase, malate dehydrogenase, ribonuclease, staphylococcal nuclease, triose phosphateisomerase, urease and yeast alcohol dehydrogenase.

In a further aspect, the present invention relates to a method wherein the desired cell after the separation, isolation and recovery is cultivated on the substantially flat non-porous body. The isolated and recovered desired cell may be removed from the substantially flat non-porous body and cultivated in an appropriate medium and container by methods well known to the person skilled in the art.

One of the advantages of the methods of the present invention is that a remarkably high recovery rate of cells is found, i.e. of the true number of desired cells present in the sample, a high number of the desired cells are isolated on the body by the method of the invention. As an example, a recovery rate in the range of 10% may be found. Preferably, within the scope of the present invention the recovery rates are in the range of 1-75%, such as in the range of 5-50%, such as 10-25%, e.g. 15-20%. These numbers are in another magnitude than the numbers found by the prior art.

A further embodiment of the present invention relates to a kit for use in any of the methods described above. The kit comprises i) a substantially flat non-porous body suitable for optical detection and analysis capable of binding a ligand regiospecifically to the surface; ii) a ligand capable of recognising and binding a desired cell type from a biological sample said ligand also being capable of binding regiospecifically to the surface of the body; iii) buffers and other reagents; iv) instructions for use of the kit according to the present invention.

The above-mentioned explanation with respect to the method for separation and isolation of a desired cell type in a biological sample also apply to the kit for use in any of the methods of the invention.

In a still further embodiment, the invention relates to the use of an assay device described above for separation and isolation of a desired cell from a biological sample.

The present invention is further described by the following examples. These examples are provided solely to illustrate the invention by reference to specific embodiments. These examplifications, while illustrating certain aspects of the invention, do not portray the limitations or circumscribe the scope of the disclosed invention. All documents mentioned herein are incorporated herein in their entirety.

### EXAMPLES

### Example 1

Optimisation of Immunochemical Parameters for Establishment of the Functional Antibody Surface

Chamber slides of glass (177380, Nunc) and pernanox (177429, Nunc) were supplied with Protein Immobilizer™ (PI) reagent (EQ0051, Exiqon) (2.5 ml for 1-chamber slides, 1 ml for 2-chamber slides) and UV irradiated for 5 minutes (the PI concentration was varied and the irradiation time were varied) (see FIG. 3). The chamber slides were washed 3 times in ultra pure water (6 ml for 1-chamber slides, 3 ml for 2-chamber slides) and dried 30 minutes at 37° C. in an incubator.

Chamber slides applied with antibody directed against mouse IgG (Fc-part), goat anti-mouse IgG (Fc-part) (Jackson, 1 5-005-071) or rabbit anti-mouse IgG (Fc-part) (Jackson, 315-005-008) were tested. The parameters included here were coupling buffer, dilution of antibody in coupling buffer, incubation time for antibody (see FIG. 6).

The optimal conditions were found to be a dilution of goat anti-mouse IgG (Fc-part) 1:800 in 0.1M sodium phosphate pH 8.0 (2.5 ml for 1-chamber slides, 1 ml for 2-chamber slides) and overnight incubation at 4° C. with agitation.

The chamber slides were washed 3 times in PBS-T (6 ml for 1-chamber slides, 3 ml for 2-chamber slides). Different drying conditions were tested. The optimal drying condition was drying with nitrogen or drying in a fume hood. The chamber slides were dried with nitrogen or in a fume hood only if stored.

The necessity for blocking was tested. Both active (with glycine, lysine) and passive (with BSA) blocking were tested. It was found that no blocking step was needed.

Chamber slides were applied with antibody directed against the cell marker CD71 (mouse anti-human CD71, MO 734, Dako). The parameters included here were antibody dilution and incubation time. The optimal conditions were found to be a dilution of mouse anti-human CD71 1:1000 in PBS-T (2.5 ml for 1-chamber slides, 1 ml for 2-chamber slides) and 1 hour incubation at room temperature with agitation.

The chamber slides were washed 3 times in PBS-T (6 ml for 1-chamber slides, 3 ml for 2-chamber slides). The chamber slides were now ready for cell application.

Chamber slides were also prepared using only antibody against CD71 antibody without antibody against IgG (Fc-part). The antibody against CD71 bound equally well to chamber slides as did the antibody against IgG (Fc-part), but in the latter cell application steps, none or very few cells could be bound to chamber slides only applied with antibody against CD71.

TABLE 1

| Parameter | Tested | Optimal condition |
|---|---|---|
| Material | Glass, Plast | Glass |
| PI concentration | 1330 to 166 μg/l | 500 μg/l |
| UV irradiation time | 1 to 10 minutes | 5 minutes |
| Anti-IgG (Fc-part) | with or without antibody | with |
| antibody species | Rabbit, goat | goat |
| coupling buffer | PBS pH 7.2 | 0.1M sodium phosphate pH 8.0 |
| | 0.1M sodium phosphate pH 8.0 | |
| | 0.1M sodium carbonate pH 8.1 | |
| | 0.1M sodium carbonate pH 9.6 | |
| concentration | 1:500, 1:800, 1:1000, 1:2000 | 1:800 |
| incubation time | 2 hours, overnight | overnight |
| Drying of chamber slides | drying with nitrogen, | drying with nitrogen, |
| | in a incubator, | in a fume hood |
| | in a fume hood | |
| Blocking | Active: glycin, lysin | not necessary |
| | Passive: 1% BSA | not necessary |
| Anti-CD71 concentration | 1:1000, 1:2000 | 1:2000 |
| incubation time | 1 hour, 2 hours | 1 hour |

Anti-IgG: Antibody against mouse IgG.
Anti-CD71: Mouse antibody against human CD-71.

### Example 2

Testing of Immunochemical Parameters in Foetal Cell Assay

Optimisation of immunochemical parameters was continuously tested in cell assays using the following cell assays:

Incubation of blood samples: Chamber slides were applied with blood samples prediluted 1:4 in PBS (2.5 ml for 1 -chamber slides, 1 ml for 2-chamber slides) and incubated for 1 hour at room temperature with agitation. The blood samples were discharged and the chambers removed from the slides. The slides were cautiously washed in three chambers containing PBS. The slides were dried for 10 to 15 minutes in a fume hood. The slides were fixed in 2% paraformaldehyd in PBS for 5 minutes. The slides were dehydrated in ethanol (62%, 96% and 99.9%) and subsequently dried for 15 minutes in a fume hood (the slides can be stored at 4° C. for several weeks at this point).

Cell Assays:

i) DAPI Staining:

One drop of DAPI stain containing Vectashield was added to each slide. Two cover slips (18×18 mm) were applied to each slide (both for 1-chamber and 2-chamber slides). The cover slips were gently pressed and the surplus of liquid was removed with a tissue. Sealing wax was applied to the edge of the cover slips in order to avoid drying out. The slides were put in a ventilated fume hood for 30 minutes prior to microscopy. The slides were now ready for microscopy.

ii) Gamma Haemoglobin Detection:

The slides were blocked in 3% BSA (in 4× SSC) with agitation for 15 minutes at room temperature. The edge of the slides was carefully dried and subsequently the edge was outlined using a Dako pen. Antibody against gamma haemoglobin; sheep anti-human gamma haemoglobin diluted 1:100 in BSA/SSC (100 μl for 2-chamber slides and 250 μl for 1-chamber slides or 2 times 100 μl for 1-chamber slides) was added to the slide. The slides were incubated for 30 minutes with agitation. The slides were washed in 4× SSC for 5 minutes, in 4× SSC-T for 5 minutes and in 4× SSC for 5 minutes with agitation. Donkey anti-sheep Ig conjugated with tetramethylrhodamine B isothiocyanate (TRITC) diluted 1:20 in BSA/SSC (100 μl for 2-chamber slides and 250 μl for 1-chamber slides or 2 times 100 μl for 1-chamber slides) was added to the slide. The slides were incubated for 30 minutes with agitation in the dark. The slides were washed in 4× SSC for 5 minutes, in 4× SSC-T for 5 minutes, in 4× SSC for 5 minutes and in 2× SSC for 5 minutes with agitation in the dark. The slides were assembled with DAPI as described above and are 30 put in a ventilated fume hood for 30 minutes prior to microscopy. The slides were now ready for microscopy.

iii) In situ Hybridisation of X- and Y-chromosomes:

The slides were placed on a heating block (37° C.). Nine μl of hybridisation mix containing XY-probes (DNA) was applied to each slide area (for 1-chamber slide 2×9 μl is applied). The X-probe was conjugated to TRITC and the Y-probe was conjugated to FITC. The slides were assembled with a cover slip and bicycle glue was applied to the edges of the cover slip to avoid evaporation. The slides were placed on a heating block (83.5° C.) for 7 minutes and subsequently at 42° C. in an incubator overnight. The glue was removed and the slides were placed in 2× SSC until the cover slips slide off (5 minutes) and the slides were washed (in the dark) in 0.4× SSC, 0.3% Nonylphenoxy Polyethoxy ethanol-40 (NP-40) at 73°+/−1° C. (water bath) for 2 minutes (use a stopwatch) and in 2× SSC, 0.1% NP-40 at room temperature for 1 minute (use a stopwatch). The slides were dried in the dark at room temperature. The slides were assembled with DAPI as described above and are put in a ventilated fume hood for 30 minutes prior to microscopy. The slides are now ready for microscopy.

The number of round DAPI positive cells, gamma-haemoglobin positive cells or XY-chromosome positive cells was used as result.

After testing the immunochemical parameters in the cell assay (see Table 2 and 3), the immunochemical parameters were determined. The above-mentioned conditions for producing antibody bound chamber slides were maintained. Glass was chosen as the most suitable material. Subsequently, chamber slides of glass were used.

TABLE 2

| | | Adult blood (control) |
|---|---|---|
| PI reagents/UV irradiation | Cord blood | (non-pregnant) |
| +PI/+UV | +++ | − |
| +PI/−UV | + | − |
| −PI/+UV | − | − |
| −PI/−UV | − | − |

Binding capacity: +++ high binding, ++ moderate binding, + weak binding, − no binding

TABLE 3

| | Binding capacity | |
|---|---|---|
| Antibody coupled to PI coated | | Adult blood (control) |
| chamber slides | Cord blood | (non-pregnant) |
| antibody against IgG (Fc-part) + | +++ | − |
| antibody against CD71 | | |
| antibody against CD71 | − | n.d. |

Binding capacity: +++ high binding, ++ moderate binding, + weak binding, − no binding
n.d.: not determined

### Example 3

Optimisation of Parameters in Foetal Cell Assays

Various cell assay parameters were optimised in order to obtain a maximum ratio between the number of bound relevant and irrelevant cells. Thus, optimum conditions for detection of relevant cells were obtained (see Table 4).

i) Dilution of Blood Samples

Dilution of cord blood samples in the range 1:1 to 1:100 was investigated. Cord blood samples were diluted in PBS and applied to chamber slides. One ml of cord blood were added to 2-chamber slides and incubated for one hour with agitation. From these tests the optimal dilution was found to be 1:4.

ii) Incubation Time of Blood Samples

The incubation time of blood samples was investigated from 1 hour of incubation to over night incubation. Cord blood was diluted 1:4 in PBS and applied to chamber slides. One ml of cord blood was added to 2-chamber slides and incubated for one hour, two hours or over night with agitation. From these tests the optimal incubation time was found to be 1 hour.

iii) Incubation in Different Media and Buffers

The following media for diluting blood samples were tested. PBS (RHONE), PBS (Oxiod), PBS (Exiqon), physiological NaCl, RPMI 1640 and Iscove media. Cord blood was diluted 1:4 in the various media/buffers and applied to chamber slides. One ml of diluted cord blood samples were added to 2-chamber slides and incubated for one hour with agitation. From these tests the optimal diluting solution was found to be PBS (RHONE).

TABLE 4

| Parameter | Tested | Optimal conditions |
|---|---|---|
| Blood samples dilution | 1:1 to 1:100 | 1:4 |
| dilution media | PBS, NaCl, culture media | PBS |
| incubation time | 1 hour, 2 hours, over night | 1 hour |
| incubation temp. | 4-8° C., 20-25° C., 37° C. | 20-25° C. |
| Blood sample application | direct, flow, batchwise, seekage | flow |
| Washing of slides | carefully in PBS | carefully in PBS |
| | 3x 2 min. in PBS with agitation | |
| | 3x 5 min. in PBS with agitation | |
| | 3x 5 min. in PBS without agitation | |
| Cytospin of bound cells | variable time and rpm | not necessary |
| Fixation of cells physical state | bound cells, cells in solution | bound cells |
| fix solution | variable solutions | 2% paraformaldehyde |
| time | 5 minutes, 10 minutes | 5 minutes |
| Detection of gamma haemoglobin | 2-layer, 3-layer method | 2-layer method |

iv) Incubation Temperature

The incubation temperature of blood samples at 4° C., 20° C. and 37° C. was tested. Cord blood was diluted 1:4 in PBS (RHONE) and applied to chamber slides. One ml of diluted cord blood samples was added to 2-chamber slides and incubated for 1 hour with agitation at 4° C., 20° C. or 37° C. From these tests the optimal incubation temperature of blood samples was found to be 20° C.

v) Washing Procedure of Chamber Slides

The chamber slides were washed after incubation with blood samples. The following washing methods were tested:

Wash carefully in PBS, i.e. the slides was dipped in PBS until all erythrocytes were washed off

Wash 3×2 min. in PBS with agitation (100 rpm).

Wash 3×5 min. in PBS with agitation (100 rpm).

Wash 3×5 min. in PBS without agitation.

From these washing procedures careful washing resulted in most bound cells. Moreover, the fewest destroyed cells were seen when using this washing method.

vi) Fixation of Cells from Cord Blood

Fixation of the cells was necessary in order to carry out the in situ hybridisation. The following different fixation methods of cord cells bound to chamber slides were tested:

Fixation in 2% paraformaldehyde for 5 minutes.

Fixation in 2% paraformaldehyde for 10 minutes.

Fixation in 5% acetic acid—11% formaldehyde for 20 minutes.

Fixation of cells in suspension was tested, i.e. fixation of cells in cord blood samples (the blood samples are diluted 1:1 in PBS and, subsequently, added to 4% paraformaldehyde 1:1), mixed for 1 hour on a blood mixer. The fixed cells were subsequently applied to the chamber slides and incubated for 1 hour with agitation. The slides were washed 3× in PBS after removal of the chamber.

After fixation of the cells, the slides were dehydrated in ethanol (62%, 96% and 99.9%) and subsequently dried for 15 minutes in a fume hood. An appropriate cell assay was performed subsequently in order to quantify the amount of bound cells. In all cell assays the cells were stained with DAPI, occasionally the cells were detected using FISH or gamma-haemoglobin detection.

It appeared from these tests that fixation in 2% paraformaldehyde for 5 minutes resulted in most bound cells and fewest destroyed cells, while fixation of the cells in suspension resulted in binding of many erythrocytes, but no erythroblasts to the chamber slides.

vii) Optimisation of Antibody Directed against Intracellular Markers

These experiments comprised optimisation of a method for detection of gamma-haemoglobin in foetal erythroblasts. A two-layer antibody method for detection of gamma-haemoglobin was applied as a substitution for a three-layer method. The two-layer and the three-layer methods are described below. The detection of gamma-haemoglobin was performed after fixation and dehydration of the bound cells on the slides.

Two-layer Antibody Method

The slides were blocked in 3% BSA (in 4× SSC) with agitation for 15 minutes at room temperature. The edge of the slides was carefully dried and subsequently the edge was outlined using a Dako pen. Antibody against gamma haemoglobin; sheep anti-human gamma haemoglobin diluted 1:100 in BSA/SSC (100 μl for 2-chamber slides and 250 μl for 1-chamber slides or 2 times 100 μl for 1-chamber slides) was added to the slides. The slides were incubated for 30 minutes with agitation. The slides were washed in 4× SSC for 5 minutes, in 4× Saline sodium citrate-0.05% Tween 20 (SSC-T) for 5 minutes and in 4× SSC for 5 minutes with agitation. Donkey anti-sheep Ig conjugated with TRITC diluted 1:20 in BSA/SSC (100 μl for 2-chamber slides and 250 μl for 1-chamber slides or 2 times 100 μl for 1-chamber slides) was added to the slides. The slides were incubated for 30 minutes with agitation in the dark. The slides were washed in 4× SSC for 5 minutes, in 4× SSC-T for 5 minutes, in 4× SSC for 5 minutes and in 2× SSC for 5 minutes with agitation in the dark. The slides were assembled with DAPI as described above and were placed in a ventilated fume hood for 30 minutes prior to microscopy. The slides were now ready for microscopy. The number of gamma-haemoglobin positive cells was used as result.

Three-layer Antibody Method

The slides were blocked in 3% BSA (in 4× SSC) with agitation for 15 minutes at room temperature. The edge of the slides was carefully dried and subsequently the edge was outlined using a Dako pen. Antibody against gamma haemoglobin; sheep anti-human gamma haemoglobin diluted 1:50 in BSA/SSC (100 μl for 2-chamber slides and 250 μl for 1-chamber slides or 2 times 100 μl for 1-chamber slides) was added to the slides. The slides were incubated for 30 minutes with agitation. The slides were washed in 4× SSC for 5 minutes, in 4× SSC-T for 5 minutes and in 4× SSC for 5 minutes with agitation.

Rabbit anti-sheep Ig diluted 1:20 in BSA/SSC (100 μl for 2-chamber slides and 250 μl for 1-chamber slides or 2 times 100 μl for 1-chamber slides) was added to the slides. The slides were incubated for 30 minutes with agitation. The slides were washed in 4× SSC for 5 minutes, in 4× SSC-T for 5 minutes, in 4× SSC for 5 minutes and in 2× SSC for 5 minutes with agitation. Swine anti-rabbit Ig conjugated to TRITC diluted 1:30 in BSA/SSC (100 μl for 2-chamber slides and 250 μl for 1-chamber slides or 2 times 100 μl for 1-chamber slides) was added to the slides. The slides were incubated for 30 minutes with agitation in the dark. The slides were washed in 4× SSC for 5 minutes, in 4× SSC-T for 5 minutes, in 4× SSC for 5 minutes and in 2× SSC for 5 minutes with agitation in the dark. The slides were assembled with DAPI as described above and were placed in a ventilated fume hood for 30 minutes prior to microscopy. The slides were now ready for microscopy. The number of gamma-haemoglobin positive cells was used as result.

In conclusion, the above mentioned tests showed that the best results were obtained with the following incubation and cell assay procedures (Table 4):

Dilution of blood samples: 1:4. Incubation time: 1 hour. Incubation buffer for blood samples: PBS. Incubation temperature when incubating blood samples: 20° C. Washing procedure: 3× washing carefully in PBS. Fixation of blood cells bound to chamber slides: 2% paraformaldehyde for 5 min. Detection of gamma haemoglobin: 2-layer method.

### Example 4

Test of Antibodies Directed Against Different Cell Markers on Cells from Core Blood and Blood from a Pregnant Woman

For the production of a functional antibody surface, antibodies against CD71, EPOr and CD36 were tested.

Chamber slides supplied with the PI functional surface were applied with antibody directed against mouse IgG (Fc-part). Goat anti-mouse IgG (Fc-part) diluted 1:800 in 0.1M sodium phosphate pH 8.0 was applied to chamber slides (1 ml for 1-chamber slides, 2.5 ml for 2-chamber slides). The chamber slides were incubated over night at 4° C. with agitation.

The chamber slides were washed 3 times in PBS-T (3 ml for 1-chamber slides, 6 ml for 2-chamber slides). Antibody against cell marker CD71, CD36 or EPOr was diluted to various concentrations in PBS-T (1 ml for 1-chamber slides, 2.5 ml for 2-chamber slides) and applied to the chamber slides. The chamber slides were incubated 1 hour at room temperature with agitation. The chamber slides were washed 3 times in PBS-T (6 ml for 1-chamber slides, 3 ml for 2-chamber slides). The chamber slides were now ready for blood sample application. All antibodies against the cell markers could be bound to the chamber slides in an optimal concentration.

The functional antibody surfaces were tested by incubation of cord blood and blood from a pregnant woman. Chamber slides were applied with blood samples prediluted 1:4 in PBS (2.5 ml for 1-chamber slides, 1 ml for 2-chamber slides) and incubated for 1 hour at room temperature with agitation. The blood samples were discharged and the chambers removed from the slides.

The slides were washed, dried, fixed and dehydrated as described in the general remarks concerning Incubation of the cells (C). The cells were detected by DAPI staining as well as in FISH using X- and Y-chromosome DNA probes and/or gamma-haemoglobin detection (see example 2, Cell assays). The number of round DAPI positive cells, XY-chromosomes positive cells and gamma-haemoglobin positive cells was used as a result.

Many foetal cells and few maternal cells were bound to chamber slides with antibody against CD71. Chamber slides with three different antibodies against EPOr (2 polyclonal and 1 monoclonal) were tested. The relevant foetal cells were bound to none of the three antibodies. By applying both polyclonal antibodies to the same slide, few foetal cells were bound. Chamber slides with antibody against CD36 did not bind foetal cells, but could bind other cell types, possibly leukocytes.

Chamber slides with antibody against CD71 were, hereby, established as the first prototype. This prototype of chamber slides was used in all subsequent tests.

### Example 5

Other Methods for Application of Blood Samples

Flow Methods

When applying blood samples, it was the intention to bind a maximum of foetal blood cells and a minimum of maternal blood cells. This was obtained by applying blood samples to chamber slides in such a manner that the blood cells got in close contact with the antibody surface, thereby optimising the affinity chromatographic binding between cells and antibody coated chamber slides. To obtain an effective binding of the blood cells, the cells had to be in close contact with the antibody surface. This can be obtained in different ways:

Method a)

Application of a blood sample in a thin stream from one end of the antibody surface under flow conditions. This means applying the blood sample drop by drop to the chamber slide.

Method b)

Application of blood samples batchwise whereby the blood sample was applied to the chamber slides in several increments.

Method c)

Application of blood samples to “non-tight” chamber slides whereby the blood sample slowly seeped through the bottom of the chamber slide. “Non-tight” chamber slides were produced by removing part of the silicone wall of the slide.

Method d)

Application of entire blood sample to a chamber slides at once (direct application)

In all three blood sample application methods, the chamber slides were prior to blood sample application coated with antibody (antibody against CD71) as described in the general remarks concerning Optimisation and testing of the immunochemical parameters (B). All blood samples were prediluted in PBS. Different dilutions were tested from 1:4 to 1: 100. Volumes from 1 ml to 50 ml were tested. All diluted blood samples were applied within 1 hour at room temperature.

As a control, the direct application method was used whereby the entire blood sample was applied to the chamber slide at the same time. One ml of blood sample (diluted 1:4 in PBS) was added to 2-chamber slides and 2.5 ml of blood sample (diluted 1:4 in PBS) was added to 1-chamber slides. The blood samples were incubated for one hour at room temperature with agitation.

The cells were detected by FISH with X- and Y-chromosome DNA probes and with DAPI staining (see example 1, Cell assays). The number of round DAPI positive cells or XY-chromosomes positive cells was used as a result.

Initial tests of methods a), b) and c) showed that application of blood by these methods can increase the number of bound cells considerably. Table 5 shows the result of a combination of methods b) and c) where the blood samples were added batchwise followed by seepage of the blood samples.

From Table 5 below it appears that a larger number of bound cells to the chamber slides was observed when the application of blood samples was done batchwise followed by seepage from the chamber slides. It is seen that a larger number of blood cells got into closer contact with the antibody surface when these methods were used. Thus, a larger affinity chromatographic binding effect of the cells to the antibody surface was obtained.

TABLE 5
Number of round DAPI positive cells per mm² (intact cells/destroyed cells)
bound selectively from blood samples.

| Blood sample | Batchwise (Method b) + c)) | Direct (Method d)) |
|---|---|---|
| Cord blood | 16/2 | 4/0 |
| Maternal blood | 26/few* | 9/0 |

*a few very destroyed cells, thus, it was difficult to state the precise cell number

In conclusion, the above-mentioned tests demonstrate that if the blood samples were added using the direct method, not all the cells will get into contact with the antibody surface on the chamber slides. By changing the application and incubation procedures, it is possible that a considerably larger number of cells will come in sufficiently close contact with the surface and, thus, the cells will have a greater possibility of binding to the antibody surface. Consequently, it is possible to bind far more relevant foetal cells from the maternal blood samples. By optimising these methods further, binding foetal cells with a higher yield is obtainable and it will be possible to bind a sufficient number of foetal cells for performing genetic diagnostics.

### Example 6

Isolation of Foetal Cells from Cord Blood Samples

Binding of foetus cells from cord blood was investigated using anti-CD71 chamber slides. Anti-CD71 chamber slides (precoated with antibody against CD71 as described in the general remarks concerning Optimisation and testing of the immunochemicalparameters (B)) were applied with blood samples prediluted 1:4 in PBS (2.5 ml for 1-chamber slides, 1 ml for 2-chamber slides) and incubated for 1 hour at room temperature with agitation.

The blood samples were discharged and the chambers removed from the slides. The slides were washed, dried, fixed and dehydrated as described in the general remarks concerning Incubation of the cells (B).

The cells were detected by DAPI staining as well as in FISH using X- and Y-chromosome DNA probes and/or with gamma-haemoglobin (see example 2, Cell assays). The number of round DAPI positive cells, XY-chromosomes positive cells and/or gamma-haemoglobin positive cells was used as a result.

i) Biological Variation of Erythroblasts in Cord Blood

The biological variation of erythroblasts in cord blood samples was investigated.

Chamber slides (precoated with antibody against CD71 as described in the general remarks concerning Optimisation and testing of the immunochemical parameters (B)) were applied with blood samples prediluted 1:4 in PBS (1 ml for 1-chamber slides) and incubated for 1 hour at room temperature with agitation.

The blood samples were discharged and the chambers removed from the slides. The slides were washed, dried, fixed and dehydrated as described in the general remarks concerning Incubation of the cells (B).

The cells were detected by DAPI staining as well as in FISH using X- and Y-chromosome DNA probes and/or with gamma-haemoglobin (see example 2, Cell assays). The number of round DAPI positive cells, XY-chromosomes positive cells and/or gamma-haemoglobin positive cells was used as a result.

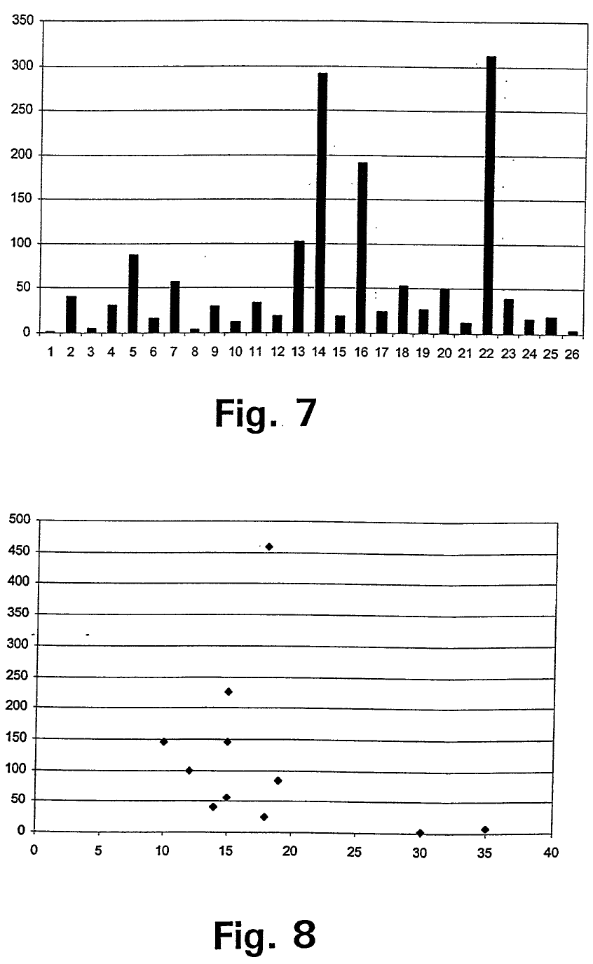

Twenty-six cord blood samples were tested. All cord blood samples were drawn the same day or the previous night prior to use. As seen in FIG. 7 there was a high variation of the number of bound DAPI positive (erythroblasts) cells.

ii) Performance of Chamber Slides

The uniformity and the reproducibility of the chamber slides binding capacity was investigated using cord blood samples.

Chamber slides (precoated with antibody against CD71 as described in the general remarks concerning Optimisation and testing of the immunochemical parameters (B)) were applied with blood samples prediluted 1:4 in PBS (1 ml for 1-chamber slides) and incubated for 1 hour at room temperature with agitation.

The blood samples were discharged and the chamber removed from the slide. The slides were washed, dried, fixed and dehydrated as described in the general remarks concerning Incubation of the cells (B).

The cells were detected by DAPI staining as well as in FISH using X- and Y-chromosome DNA probes and/or with gamma-haemoglobin (see example 2, Cell assays). The number of round DAPI positive cells, XY-chromosomes positive cells and/or gamma-haemoglobin positive cells was used as a result.

The binding of cells from different areas of the same slides showed that the cells were very uniformly distributed throughout the chamber slide, see Table 6. As seen in Table 6, binding of cells from the same blood sample on different slides, were very reproducible.

TABLE 6
The uniformity and reproducibility of chamber slides incubated
with a cord blood sample. The cell count per mm² of DAPI positive
cells. The number of DAPI positive cells were counted in different
areas of the chamber slides.

| Slide | DAPI staining |
|---|---|
| Slide 1 | 36, 46, 40, 35, |
| | 39, 32, 26 |
| Slide 2 | 47 |
| Slide 3 | 37 |

iii) Recovery of Cells from Cord Blood Samples

Incubation of cord blood, 1 ml diluted 1:4 (200 μl diluted in 800 μl PBS) on 2-chamber chamber slides, was incubated on 484 mm². In general 40-50 DAPI positive cells (erythroblasts) per mm² were bound (FIG. 6). This is 19.360-24.200 cells per slide and 96.800 -121.000 cells per ml of cord blood. Normally there are 1×10⁶ erythroblasts per ml. This calculates to a recovery of 9.7 to 12. 1%, which is a very good recovery.

In conclusion, the above-mentioned tests showed a high biological variation of cells in cord blood. This variation is not caused by a variation of bound cells within the same slide, as it appeared when testing the uniformity of the bound cells on the surface that they were bound relatively uniformly over the entire chamber slide surface. Moreover, it appeared that the reproducibility of the chamber slides is high, as different slides incubated with the same sample show similar results. It also appeared that the recovery was relatively high considering that in these tests, the blood samples were applied in accordance with the direct method.

### Example 7

Isolation of Cells from Foetus Blood Samples during Different Age Periods of the Foetus

Blood samples from foetuses were drawn after provocated aaboition. Foetuses of the age 10 to 14 weeks are extremely small and are therefore very difficult to draw a blood sample from. Blood samples from foetuses during this age are always diluted beforehand and it was therefore very difficult to estimate the exact concentration of the samples.

Chamber slides (precoated with antibody against CD71 as described in the general remarks concerning Optimisation and testing of the immunochemical parameters (B)) were applied with foetus blood samples preferentially prediluted 1:4 in PBS (1 ml for 1-chamber slides) and incubated for 1 hour at room temperature with agitation.

The foetus blood samples were discharged and the chamber removed from the slide. The slides were washed, dried, fixed and dehydrated as described in the general remarks concerning Incubation of the cells (B).

The cells were detected by DAPI staining as well as in FISH using X- and Y-chromosome DNA probes and/or with gamma-haemoglobin (see example 2, Cell assays). The number of round DAPI positive cells, XY-chromosomes positive cells and/or gamma-haemoglobin positive cells was used as a result

From FIG. 8 it is seen that there was a large variation of the amount of foetus cells bound to chamber slides during different age periods (DAPI staining). As seen from FIG. 8 the functional surface has a very high binding capacity of foetus cells at this very relevant foetus age.

Most abundant were foetal cells from foetuses aged 10 to 18 weeks. This can be due to a relatively high concentration of CD7 1 positive cells or to a higher density of CD71 on the cell surface.

Foetus aged 30-35 weeks bound very few relevant cells, whereas foetus aged 40 weeks (cord blood, taken after birth) again bound many cells. This showed that the amount of foetal cells in the relevant pregnancy period (10-14 weeks) that expressed CD71 on the cell surface is high or/and that the density of CD71 on the cell surface was higher at this period.

The detected foetal cells were found to be erythroblasts, due to their positive response for gamma-haemoglobin. These cells were detected with antibody directed against gamma-haemoglobin followed by incubation with TRITC conjugated antibody against Ig.

### Example 8

Isolation of Foetal Cells from Maternal Blood Samples

i) Maternal Blood Samples Drawn during Pregnancy

Blood samples from two pregnant women (W1 and W2) were drawn at different periods during their pregnancy. Blood samples were drawn with 2-week intervals over a 2-month period starting at week 9 (W1) and 12 (W2).

Chamber slides (precoated with antibody against CD7 1 as described in the general remarks concerning Optimisation and testing of the immunochemical parameters (B)) were applied with maternal blood samples prediluted 1:4 in PBS (2 times 5 ml for 1-chamber slides) and incubated for 1 hour at room temperature with agitation.

The maternal blood samples were discharged and the chamber removed from the slide. The slides were washed, dried, fixed and dehydrated as described in the general remarks concerning Incubation of the cells (B).

The cells were detected by DAPI staining as well as in FISH using X- and Y-chromosome DNA probes and/or gamma-haemoglobin staining (see example 2, Cell assays). The number of round DAPI positive cells, XY-chromosomes positive cells and/or gamma-haemoglobin positive cells was used as a result.

One XY-positive cell (blood sample week 12 from W1) and one gamma-haemoglobin positive cell (blood sample week 15 from W2) was detected in blood samples from the two women. At time for labour W1 gave birth to a boy and W2 gave birth to a girl.

ii) Maternal Blood Samples from Pregnant Women Prior to a CVS Test

a) Blood Samples were Drawn from Pregnant Women in Connection with CVS Tests

These blood samples were drawn prior to a CVS test from pregnant women. Blood samples from eight women expecting boys and five women expecting girls were tested. The maternal blood samples were drawn at 10 to 12 weeks of pregnancy.

Chamber slides (precoated with antibody against CD71 as described in the general remarks concerning Optimisation and testing of the immunochemical parameters (B)) were applied with blood samples from pregnant women prediluted 1:4 in PBS (2×5 ml in 1-chamber slides) and incubated for 1 hour at room temperature with agitation.

The maternal blood samples were discharged and the chamber removed from the slide. The slides were cautiously washed in three chambers containing PBS. The slides were washed, dried, fixed and dehydrated as described in the general remarks concerning Incubation of the cells (B).

The cells were detected by DAPI staining as well as in FISH using X- and Y-chromosome DNA probes and/or gamma-haemoglobin staining (see example 2, Cell assays). The number of round DAPI positive cells, XY-chromosomes positive cells and/or gamma-haemoglobin positive cells was used as a result.

In this test one XY-positive cell was found in one maternal blood sample of the eight blood samples tested (blood samples from women expecting a boy) and one gamma-haemoglobin positive cell was detected in one blood sample of the five blood samples tested (blood samples from women expecting a girl).

It is therefore possible to detect foetal cells even in a very small blood sample (2 ml) using this application method (direct method).

b) Blood Samples were Drawn from Pregnant Women in Connection with CVS Tests.

These blood samples were drawn prior to a CVS test from pregnant women. Twenty-seven blood samples drawn from pregnant women prior to CVS sampling were investigated for binding of maternal cells.

Chamber slides (precoated with antibody against CD71 as described in the general remarks concerning Optimisation and testing of the immunochemical arameters (B)) were applied with blood samples from pregnant women prediluted 1:4 in PBS (2×5 ml in 1-chamber slides) and incubated for 1 hour at room temperature with agitation.

The maternal blood samples were discharged and the chamber removed from the slide. The slides were washed, dried, fixed and dehydrated as described in the general remarks concerning Incubation of the cells (B).

The cells were detected by DAPI staining as well as in FISH using X- and Y-chromosome DNA probes and/or gamma-haemoglobin staining (see example 2, Cell assays). The number of round DAPI positive cells, XY-chromosomes positive cells and/or gamma-haemoglobin positive cells was used as a result.

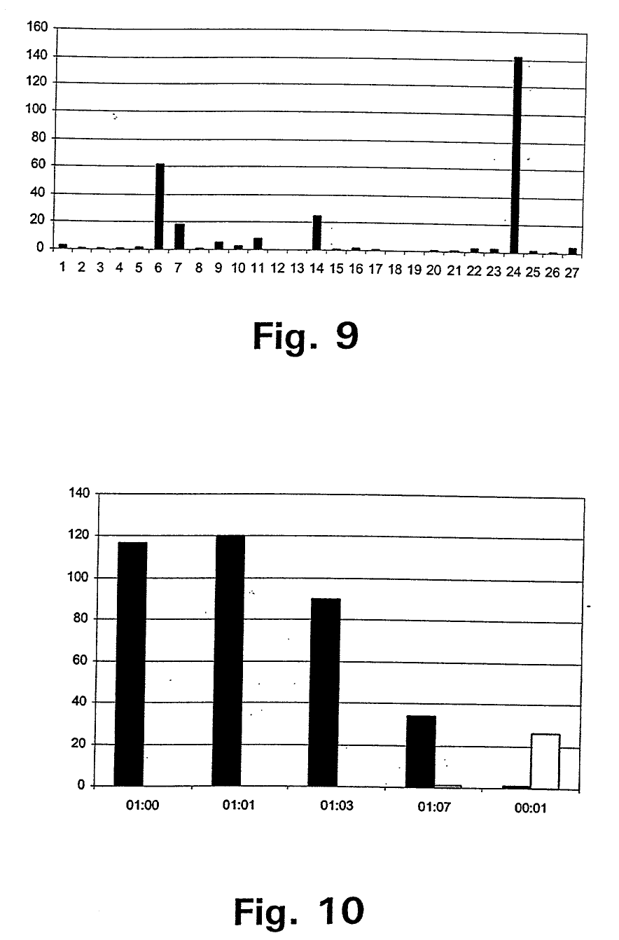

Very few samples showed any substantial binding (4 samples with >10 cells per mm²) (FIG. 9). Even in these 4 samples the cells only occupied a minor area of the slide, thereby allowing extensive space for binding of the foetal cells that could be present in the samples.

iii) Blood Samples Taken after Birth

Blood samples were drawn from women shortly after giving birth.

Chamber slides (precoated with antibody against CD71 as described in the general remarks concerning Optimisation and testing of the immunochemical parameters (B)) were applied with blood samples from pregnant women prediluted 1:4 in PBS (2×5 ml in 1-chamber slides) and incubated for 1 hour at room temperature with agitation.

The maternal blood samples were discharged and the chamber removed from the slide. The slides were washed, dried, fixed and dehydrated as described in the general remarks concerning Incubation of the cells (B).

The cells were detected by DAPI staining as well as in FISH using X- and Y-chromosome DNA probes and/or gamma-haemoglobin staining (see example 2, Cell assays). The number of round DAPI positive cells, XY-chromosomes positive cells and/or gamma-haemoglobin positive cells was used as a result.

In six of the blood samples tested, XY-chromosome positive cells were found in five blood samples. Samples of one to five ml of blood were used. In the five blood samples between 1 to 23 XY-chromosome positive cells were detected. This shows that it is possible to detect foetal cells in maternal blood even with very small samples.

From the above-mentioned tests in section i) to iii), it may be concluded that it is possible to isolate foetal cells from maternal blood samples on anti-CD71 chamber slides. This was done using the direct method. It appeared that from blood samples drawn after delivery foetal cells can be isolated more frequently than from blood samples drawn in the first or second trimester. Thus, it is seen that it is possible to isolate foetal cells, even in blood samples with a lot of granulocytes bound to the chamber slides.

### Example 9

Cord Blood Samples Titrated in the Respective Maternal Blood Samples

i) 2-fold Titration of Cord Blood in Maternal Blood

Cord blood from a newborn boy was 2-fold titrated in maternal blood. Blood samples were diluted 1:4 in PBS prior to titration.

Chamber slides (precoated with antibody against CD71 as described in the general remarks concerning Optimisation and testing of the immunochemical parameters (B)) were applied with the titrated blood samples prediluted 1:4 in PBS (1 ml in 2-chamber slides) and incubated for 1 hour at room temperature with agitation.

The cord blood/maternal blood samples were discharged and the chamber removed from the slide. The slides were cautiously washed in three chambers containing PBS. The slides were washed, dried, fixed and dehydrated as described in the general remarks concerning Incubation of the cells (C).

The cells were detected by DAPI staining as well as in FISH using X- and Y-chromosome DNA probes and/or gamma-haemoglobin staining (see example 2, Cell assays). The number of round DAPI positive cells, XY-chromosomes positive cells and/or gamma-haemoglobin positive cells was used as a result.

Male cells were detected as cells positive for X-chromosome and Y-chromosome in situ hybridisation. See FIG. 10. One XY-chromosome positive cell was detected in the maternal blood sample without the additional cord blood.

ii) 4-fold Titration of Cord Blood in Maternal Blood

Furthermore, cord blood from a new-born boy was 4-fold titrated in maternal blood. Blood samples were diluted 1:4 in PBS prior to titration. One ml of blood sample was applied to 2-chamber chamber slides.

The cord blood/maternal blood samples were discharged and the chamber removed from the slide. The slides were washed, dried, fixed and dehydrated as described in the general remarks concerning Incubation of the cells (C).

The cells were detected by DAPI staining as well as in FISH using X- and Y-chromosome DNA probes and/or gamma-haemoglobin staining (see example 2, Cell assays). The number of round DAPI positive cells, XY-chromosomes positive cells and/or gamma-haemoglobin positive cells was used as a result.

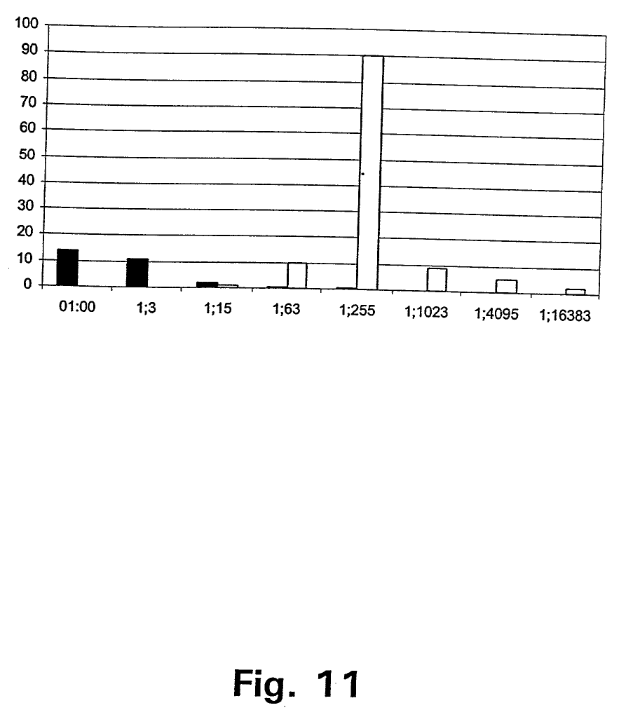

Male cells were detected using X-chromosome and Y-chromosome in situ hybridisation. See FIG. 11.

As seen in FIGS. 10 and 11 the binding of foetal cells to chamber slides was very selective compared to binding of maternal cells. Maternal cells were not bound to chamber slides until the foetal cells were applied in a high dilution (1:63). Foetal cells could be detected in a solution diluted 1:255 by manual microscopy. However, it cannot be ruled out that foetal cells are present in the samples at a higher dilution, as it can be difficult to manually detect the individual XY-positive cells among several thousand maternal cells.

### Example 10

Binding of Foetal Cells from Cord Blood was Investigated using Slides with CD36 Antibodies

Chamber slides (precoated with anti-CD36) are applied with blood samples prediluted 1:4 in PBS (2.5 ml for 1-chamber slides, 1 ml for 2-chamber slides) and incubated for 1 hour at room temperature with agitation.

The blood samples were discharged and the chambers removed from the slides. The slides were washed, dried, fixed and dehydrated as described in the general remarks concerning Incubation of the cells (C). The cells were detected by DAPI staining and with FISH using X- and Y-chromosome DNA probes and/or with gamma-haemoglobin (see example 1, cell assays). The number of round DAPI positive cells, XY-chromosome positive cells and/or gamma-haemoglobin positive cells was used as result.

A large number of foetal cells were bound. These cells were DAPI positive and XY-chromosome positive. The cells were gamma-haemoglobin negative. The cells were not erythroblasts, but were probably lymfocytes. This shows that the chamber slides are able to bind other subpopulations in blood samples than erythroblasts using other cell markers. Isolation of lymfocytes is though not relevant, as they do not have specific foetal cell markers for identification.

What is claimed is:

1. A method for separation and isolation of a desired cell type in a biological sample by binding said cell to a ligand specifically recognising said cell, the method comprising binding the ligand regio-specifically to the surface of a substantially flat non-porous body suitable for optical detection and analysis.

2. A method according to claim 1, wherein the desired cell is a foetal cell, blood cell, cancer cell, stem cell, malignant cell, mutated cell, transfected cell, genetically modified cell, spermatocyte or cell from a cell line.

3. A method according to claim 1 or 2, wherein the desired cell is a cell at a specific differential stage.

4. A method according to claim 3, wherein the desired cell is a blood cell at a specific differential stage of the myeloid or lymphoid cell lineage.

5. A method according to any of claims 2-4, wherein the stem cell is a hematopoietic stem cell selected from the group consisting of blast cell forming unit (BFU-E), colony forming unit granulocytes/monocytes (CFU-GM) and colony forming unit megacaryocytes (CFU-MG).

6. A method according to claim 1, wherein the ligand is a protein, polypeptide, hormone or polysaccharide.

7. A method according to claim 1, wherein the ligand is an antibody, a receptor, an enzyme or a lectin.

8. A method according to claim 7, wherein the antibody is a monoclonal antibody.

9. A method according to claim 7, wherein the antibody is a polyclonal antibody.

10. A method according to any of claims 6-9, wherein the ligand is specifically binding a cell surface marker, cell surface receptor or a MHC cell surface marker on the respective cell.

11. A method according to claim 7, wherein the ligand is an antibody specifically binding a cell surface marker, cell surface receptor or a MHC cell surface marker on the respective cell.

12. A method according to claim 10 or 11, wherein the cell surface marker is a CD-antigen.

13. A method according to claim 10 or 11, wherein the cell surface receptor is EPOr or a T-cell receptor.

14. A method according to claim 10 or 11, wherein the MHC cell surface marker is an HLA-antigen or another tissue-typing antigen.

15. A method according to any of the preceding claims, wherein the biological sample is from an individual.

16. A method according to any of the preceding claims, wherein the biological sample is from a mammal including a mouse, rat, guinea pig, pig, rabbit, monkey, cat, dog, horse, cow, goat, sheep and a human.

17. A method according to any of the preceding claims, wherein the biological sample for detection of the desired cell is from an apparently healthy individual.

18. A method according to any of the preceding claims, wherein the biological sample for detection of the desired cell is from an individual considered to have a risk of suffering from a disease e.g. cancer, arthritis, allergy, inflammatoric disorders or neurological disorders.

19. A method according to any of the preceding claims, wherein the biological sample for detection of the desired cell is from an individual suffering from a disease e.g. cancer, arthritis, allergy, inflammatoric disorders or neurological disorders.

20. A method according to any of the preceding claims, wherein the biological sample for detection of the desired cell is from an individual who has suffered from a disease e.g. cancer, arthritis, allergy, inflammatoric disorders or neurological disorders and is therefore of interest to determine whether the desired cell type is present in the sample.

21. A method according to any of the preceding claims, wherein the biological sample for detection of the desired cell is from a dead individual.

22. A method according to any of the preceding claims, wherein the biological sample is selected from the group consisting of blood, buffy coat, cerebrospinal fluid, urine, salvia and other biological fluid samples.

23. A method according to any of claims 1-21, wherein the biological sample is a tissue sample.

24. A method according to any of the preceding claims, wherein the substantially flat non-porous body is made from plastic, glass, silicone, silicone oxide (silica) or a composite material thereof.

25. A method according to claim 24, wherein the surface from the substantially flat non-porous body is a polystyrene, polyethylene, polyvinylacetate, polyvinylchloride, polyvinylpyrrolidone, polyacrylonitrile, polymethyl-methacrylatepentylene, polyester, polypropylene, polyvinylidendifluoride, polycarbonate or Topas surface.

26. A method according to claim 24 or 25, wherein the substantially flat non-porous body is selected from a sheet, a film, a disc, a plate, a ring, a rod, a tube, a tray, a microtiter plate, a cluster tray, a stick, a slide and a microscope slide.

27. A method according to any of the preceding claims wherein the desired cell after the separation, isolation and recovery is cultivated on the substantially flat non-porous body.

28. A method according to claim 27, wherein the isolated and recovered desired cell is removed from the substantially flat non-porous body.

29. A method according to claim 28, wherein the removed cell is cultivated in an appropriate medium and container.

30. A kit for use in any of the methods in any of the preceding claims, the kit comprising
i) a substantially flat non-porous body suitable for optical detection and analysis capable of binding a ligand regiospecifically to the surface
ii) a ligand capable of recognising and binding a desired cell type from a biological sample said ligand also capable of binding regiospecifically to the surface of the body
iii) buffers and other reagents
iv) instructions for use of the kit

31. A kit for use in any of the methods in any of the preceding claims, the kit comprising
i) a substantially flat non-porous body suitable for optical detection and analysis with a ligand regiospecifically bound to the surface said ligand capable of recognising and binding a desired cell type from a biological sample
ii) buffers and other reagents
iii) instructions for use of the kit

32. A kit according to claim 30 and 31, wherein the desired cell is a foetal cell, cancer cell, stem cell, malignant cell, mutated cell, transfected cell, genetically modified cell, blood cell, spermatocyte, or a cell from a cell line.

33. A kit according to claim 30 and 31, wherein the desired cell is a cell at a specific differential stage of the foetal cells, blood cells, cancer cells, stem cells, malignant cells, mutated cells, transfected cells, genetically modified cells, spermatocytes or cells from a cell line.

34. A kit according to claim 30 and 31, wherein the stem cell is a hematopoietic stem cell selected from the group consisting of blast cell forming unit (BFU-E), colony forming unit granulocytes/monocytes (CFU-GM) and colony forming unit megacaryocytes (CFU-MG).

35. A kit according to claim 30 and 31, wherein the ligand is a protein, polypeptide, hormone or polysaccharide.

36. A kit according to claim 30 and 31, wherein the protein ligand is an antibody, a receptor, an enzyme or a lectin.

37. A kit according to claim 30 and 31, wherein the antibody ligand is a monoclonal antibody.

38. A kit according to claim 30 and 31, wherein the ligand is a polyclonal antibody.

39. A kit according to claim 30 and 31, wherein the ligand is specifically binding a cell surface marker, cell surface receptor or a MHC cell surface marker on the respective cell.

40. A kit according to claim 30 and 31, wherein the ligand is an antibody specifically binding a cell surface marker, cell surface receptor or a MHC cell surface marker on the respective cell.

41. A kit according to claim 39 and 40, wherein the surface marker is a CD-antigen.

42. A kit according to claim 39 and 40, wherein the cell surface receptor is EPOr or the T-cell receptor.

43. A kit according to claim 39 and 40, wherein the MHC cell surface marker is a HLA-antigen or another tissue typing antigen.

44. A kit according to claim 30 and 31, wherein the biological sample is from an individual.

45. A kit according to claim 44, wherein the individual is a mammal including a mouse, rat, guinea pig, pig, rabbit, monkey, cat, dog, horse, cow, goat, sheep and a human.

46. A kit according to claim 30 and 31, wherein the sample is from an apparently healthy individual.

47. A kit according to claim 30 and 31, wherein the individual is considered to have a risk of suffering from a disease e.g. cancer, arthritis, allergy, inflammatoric disorders or neurological disorders

48. A kit according to claim 30 and 31, wherein the individual is suffering from a disease e.g. cancer, arthritis, allergy, inflammatoric disorders or neurological disorders

49. A kit according to claim 30 and 31, wherein the individual has suffered from a disease e.g. cancer, arthritis, allergy, inflammatoric disorders or neurological disorders neurological disorders and is of interest to determine whether the desired cell is present in the sample.

50. A kit according to claim 30 and 31, wherein the biological sample is selected from the group consisting of blood, buffy coat, cerebrospinal fluid, urine, salvia, tissue samples and other biological fluid samples.

51. A kit according to claim 30 and 31, wherein the biological sample for detection of the desired cell is from a dead individual.

52. Use of an assay device described in any of the preceding claims for separation and isolation of a desired cell from a biological sample.
